(19) United States
(12) Patent Application Publication
(10) Pub. No.: US 2005/0059950 A1
(43) Pub. Date: Mar. 17, 2005

(54) Absorbent garments with extendable side panels

(76) Inventor: Kathy Murguly, Hillsborough, NJ (US)

Correspondence Address: ROPES & GRAY LLP, ONE INTERNATIONAL PLACE, BOSTON, MA, 02110-2624, US

(21) Appl. No.: 10/942,471

(22) Filed: Sep. 16, 2004

Related U.S. Application Data
(60) Provisional application No. 60/503,532, filed on Sep. 16, 2003.

(51) Int. Cl. A61F 13/15; A61F 13/20

(52) U.S. Cl. 604/387

(57) ABSTRACT

The present invention discloses an absorbent article with extendable side panels. The side panel comprises a sheet of skin-friendly material, each having a first edge and a second edge extending in a lengthwise direction, and a third edge and a fourth edge each extending in a widthwise direction. The side panel also include fasteners, positioned along the third edge, adapted to affix said side panel to the front or back of the ears of a conventionally designed absorbent article. The fourth edge of the side panel is attached to the left and right ear of the absorbent article. The extendable side panels allow for an absorbent garment that provides for a wide range of circumferences.

### CROSS-REFERENCE TO RELATED APPLICATIONS

This patent application claims the benefit of, and incorporates by reference in its entirety, prior provisional patent application U.S. Provisional Patent Application No. 60/503,532, filed Sep. 16, 2003.

### BACKGROUND OF THE INVENTION

The marketplace provides various size adult absorbent garments (small, medium, large and extra large) for incontinent patients. The extra large size has the largest waist circumference of up to a maximum of 63 to 65 inches. Unfortunately, there are some incontinent patients who require garments that can provide for a waist circumference greater than 65 inches. For these individuals, there are no commercially available products that meet their needs.

Manufacturing adult absorbent garments with waist circumferences greater than 65 inches would require specialized, expensive production equipment, capable of handling extremely wide and heavy material rolls. They would also require wider adhesive application systems, wider rotary cutting knives, larger packaging equipment, among other things. When taking all of this into consideration, along with the fact that there are not many incontinent users of this large sized product, one can see why there are no manufacturers of this type of product today. A commercial need exists to provide a product that is a cost effective alternative.

### SUMMARY OF THE INVENTION

The present invention discloses an absorbent article with extendable side panels. The side panels comprise a sheet of skin-friendly material, each having a first edge and a second edge extending in a lengthwise direction, and a third edge and a fourth edge extending in a widthwise direction. The side panels also include fasteners, preferably positioned along the third edge, adapted to affix said side panels to the front or back of the ears of a conventionally designed absorbent article. The fourth edge of the side panels are bonded to the left and right ear of the absorbent article. The extendable side panels allow for an absorbent garment that provide for a wide range of circumferences.

In another embodiment, the present invention discloses an absorbent article with a single extendable side panel. The side panel comprises a sheet of skin-friendly material, having a first edge and a second edge extending in a lengthwise direction, a third edge and a fourth edge extending in a widthwise direction, and a middle portion positioned between the first and second edges. This side panel includes fasteners, positioned along the third edge and middle portion, adapted to affix said side panels to the front portion of the absorbent garment and one of its back ears. The fourth edge of the side panel is bonded to the left and right ear of the absorbent article. The extendable side panel allows for an absorbent garment that provides for a wide range of circumferences.

### BRIEF DESCRIPTION OF THE DRAWINGS

FIG. 1 is a top view of one embodiment of the present invention comprising rectangular-shaped side panels that are attached to the absorbent garment.

FIG. 2 is a top view of another embodiment of the present invention comprising trapezoidal (taper-shaped) side panels that are attached to the absorbent garment.

FIG. 3 is a top view of the embodiment depicted in FIG. 2 with the side panels folded out.

FIG. 4A is a cross-sectional view showing how one embodiment of the present invention can be constructed, where the side panels are attached to the top surface (body side facing) of the absorbent garment, and where the side panel includes a fastening tape for attachment.

FIG. 4B is a cross-sectional view showing how another embodiment of the present invention can be constructed, where the side panels are attached to the back surface (outside facing) of the absorbent garment and then folded over the top surface of the garment (body facing surface), and where the side panel includes fastening tape for attachment.

FIG. 5A is a cross-sectional view showing how another embodiment of the present invention can be constructed, where the side panels are attached to the top surface (body side facing) of the absorbent garment, and where the side panel includes a positioning adhesive for attachment.

FIG. 5B is a cross-sectional view showing how another embodiment of the present invention can be constructed, where the side panels are attached to the back surface (outside facing) of the absorbent garment and then folded over the top surface (body facing surface), and where the side panel includes positioning adhesive for attachment.

FIG. 6A is a top view of another embodiment of one side panel (prior to bonding to the absorbent garment) of the present invention, showing a central elastic section and two non-elastic sections. The side panel includes a positioning adhesive and a release liner as a means of attachment.

FIG. 6B is a cross-sectional view showing how one side panel of the present invention (prior to bonding to the absorbent garment) can be constructed.

### DETAILED DESCRIPTION OF THE INVENTION

The present invention provides a means for incontinent bariatric users to use adult brief products containing extendable side panels that can cover an extremely wide range of waist circumferences. The present invention also provides a more practical and cost-effective means to manufacture extremely wide products using current conventional high-speed manufacturing machines that currently produce commercial-sized absorbent garments (such as Small, Medium, Large and Extra Large sizes).

The absorbent garment of the present invention can be made by maintaining the current width dimensions on the conventional manufacturing machines, and modifying the garments to allow the attachment of the side panels to the outer edge (top or bottom) of the absorbent garment's ears, i.e. “cut and place”. These products can replace current commercially available products (Small, Medium, Large and Extra Large sizes), and provide the potential to expand the product for patients requiring Extra and Extra Large (XXL) sizes, i.e. those sizes extending beyond 65 inches in waist circumference. The maximum circumference attainable according to the present invention is not limited and depends only on the width of the side panels used.

The term “bonding” as used herein refers to the method used to bond or attach the side panels of the present invention to the ears or the sides of an absorbent garment. Such bonding can be accomplished by various methods including, without limitation, ultrasonic bonding, heat sealing, adhesive or cohesive attachment, mechanical attachment (hook & loop), stitching, taping or any other methods well known in the art. It is understood that this “bonding” does not have to be permanent and can be accomplished as suggested by adhesive or mechanical attachment, which allows for the side panels to be detached and reattached as needed.

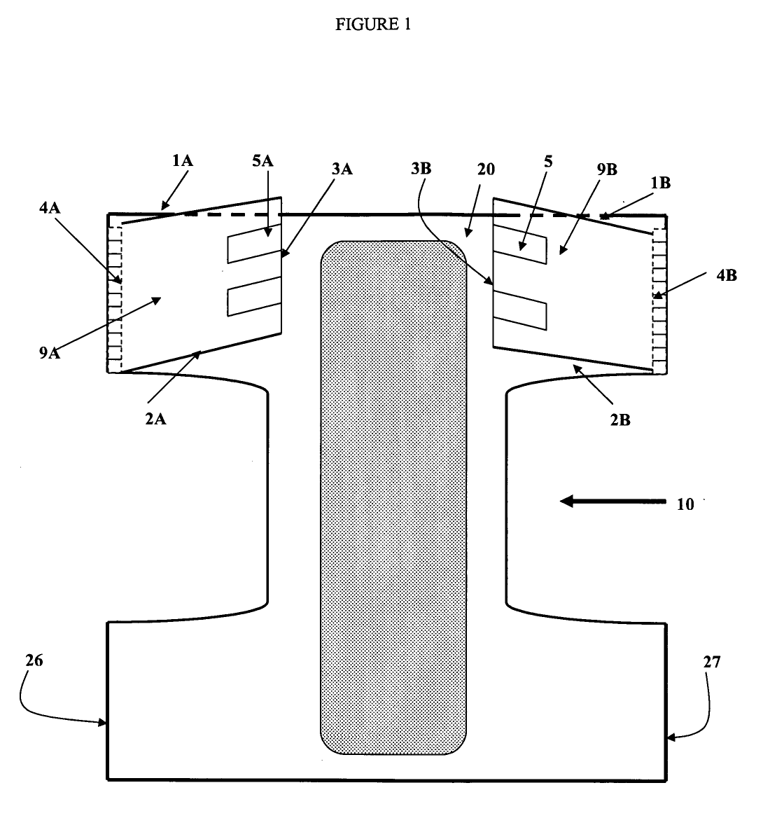

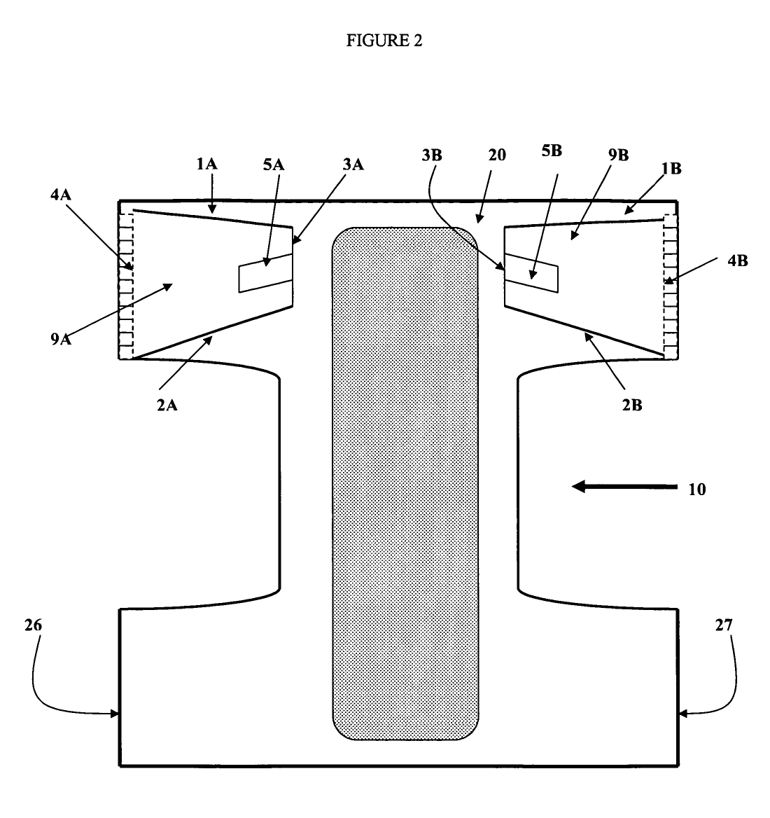

FIGS. 1, 2 and 3 illustrate side panels 9A and 9B (hereinafter “9”) of the present invention bonded to top surface 20 of absorbent garment 10. It is understood that these panels can be rectangular, as shown in FIG. 1, or have other shapes such as the trapezoidal panels shown in FIG. 2. In these Figures, the side panels 9 comprise a sheet of skin-friendly material having first edges 1A and 1 B (hereinafter “1”) and second edges 2A and 2B (hereinafter “2”), extending in a lengthwise direction, third edges 3A and 3B (hereinafter “3”), and fourth edges 4A and 4B (hereinafter “4”), extending in a widthwise direction. Side panels 9 are bonded to the right edge and left edge (or ear) of absorbent garment 10 by bonding the side panels along fourth edges 4 to the left and right edges. Side panels 9 include fastening/release tapes 5A and 5B (hereinafter “5”), which are positioned on third edges 3 as shown in FIGS. 1 and 2. Fastening tapes 5 allow for attachment to left edge 26 and right edge 27 of the front portion of the garment. In the alternative, depending on the orientation desired, the panels can be attached to the front portion of the garment to allow the attachment of the front portion to the back portion.

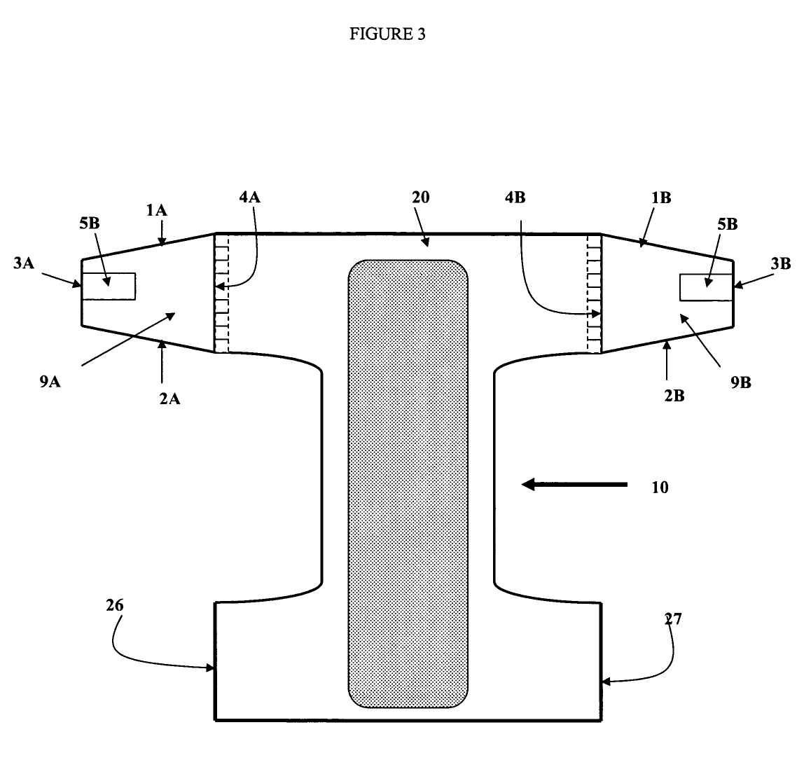

Note that the taper-shaped side panel shown in FIG. 2 can accommodate a single fastening tape on “narrower” third edges 3. FIG. 1 comprises a rectangular-shaped side panel, which may require more than one fastening tape on third edges 3. Fastening tapes 5 can be applied in a single strip or intermittently, or in any other manner as long as it allows the side panel to sufficiently attach to the absorbent garment. FIG. 3 illustrates the top view of how the absorbent garment would appear once side panels were extended out from the ears. As illustrated, fourth edge 4 of the side panel is about the same width as the ear, but it can be narrower, to save on material costs and therefore be more manufacturing friendly. However, the width of this edge should not be so narrow as to cause the absorbent garment to possibly shift on the wearer while being worn.

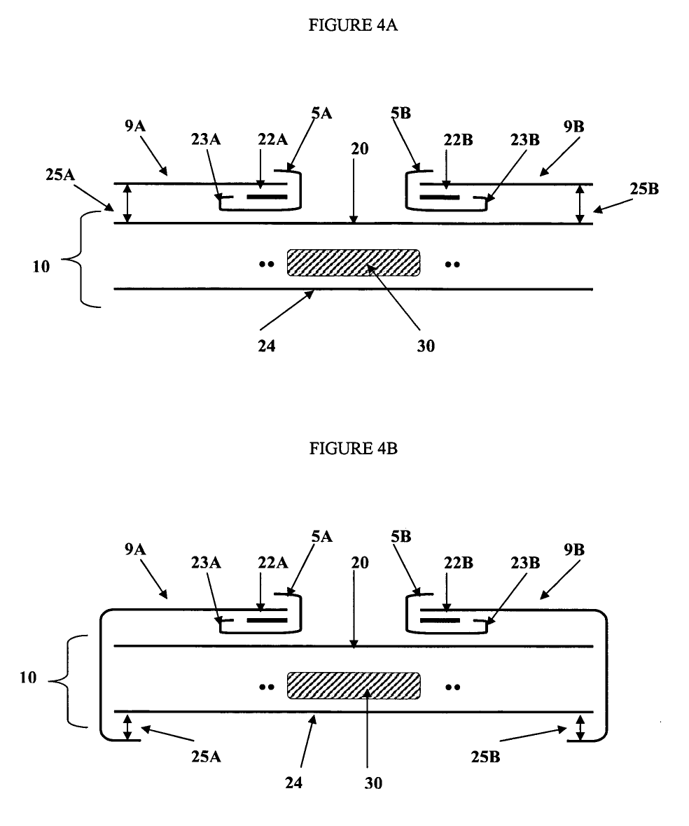

FIG. 4A and FIG. 4B illustrate two possible ways to construct the absorbent garment with extendable side panels of the present invention. Both Figures illustrate top surface 20, back surface 24, absorbent core 30, and fastening/release tape 5 with a finger tab 23A and 23B (hereinafter “23”), which allows ease of removal to expose adhesive 22A and 22B (hereinafter “22”) to allow for attachment. FIG. 4A shows fourth edges 4 of the side panel bonded to the right and left ear of the absorbent garment by bond 25A and 25B (hereinafter “25”). FIG. 4A depicts the side panel bonded to top surface 20. In an alternative embodiment shown in FIG. 4B, the side panel is bonded to back surface 24. Side panels 9 unite the back portion of absorbent garment 10 with the left edge and the right edge of the front portion of the garment. In the alternative, depending on the orientation desired, the panels can be attached to the front portion to allow the attachment of the front portion to the back portion.

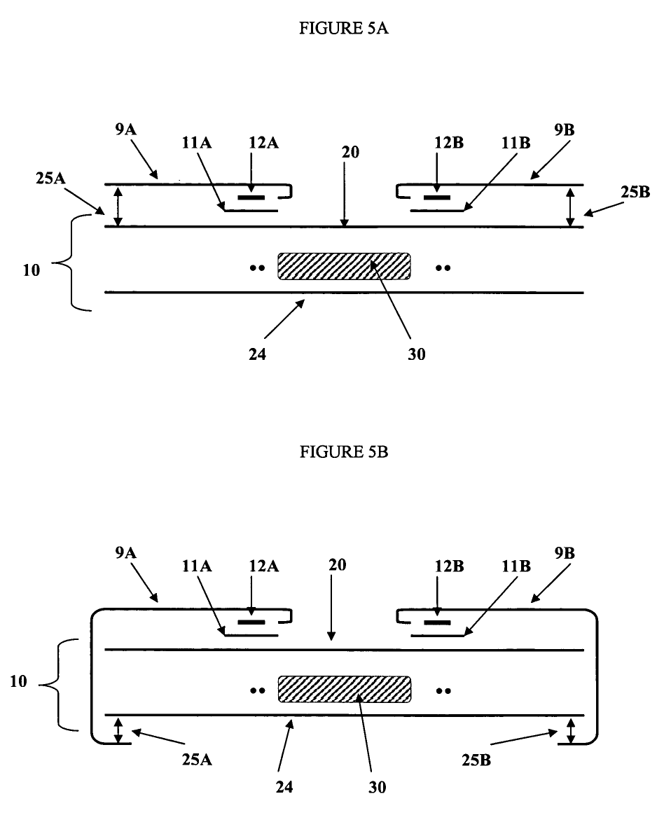

FIG. 5A and FIG. 5B illustrate two other ways to construct the absorbent garment with extendable side panels of the present invention with positioning adhesives. Both Figures illustrate a fastener comprising release liner 11A and 11B, and positioning adhesive 12A and 12 B, which allow for attachment. FIG. 5A shows the fourth edge of the side panel bonded to the right and left ear of the absorbent garment by bonds 25. FIG. 5A depicts the side panel bonded to top surface 20. In an alternative embodiment shown in FIG. 5B, the side panel is bonded to back surface 24.

The sheet used to make the side panel of the present invention can be made of various skin-friendly materials. The term “skin-friendly” in the context of the invention is intended to mean any comfortable material, including breathable material, that will mitigate any kind of discomfort to the wearer when in contact with their skin. One such material is poly laminates commercially available from Clopay Plastic Products of Mason, Ohio. Another acceptable material is a non-woven fabric, such as SBPP (Spundbond Polypropylene) or SMS (Spunbond-Meltblown-Spunbond). Such non-woven materials are commercially available from SAAF Advanced Fabrics of Al-Ahsa, Saudi Arabia. Yet other acceptable materials are other non-woven fabrics such as ABPET (Adhesive Bonded Polyester), TABICO (Through Air Bonded Bi-Component Fiber), TBPP (Thermal Bonded polypropylene), spunlace fibers, hydro-entangled fibers, needle-punched fibers, airlaid composites and woven fabrics. These materials are commercially available from PGI Nonwovens of Mooresville, N.C.

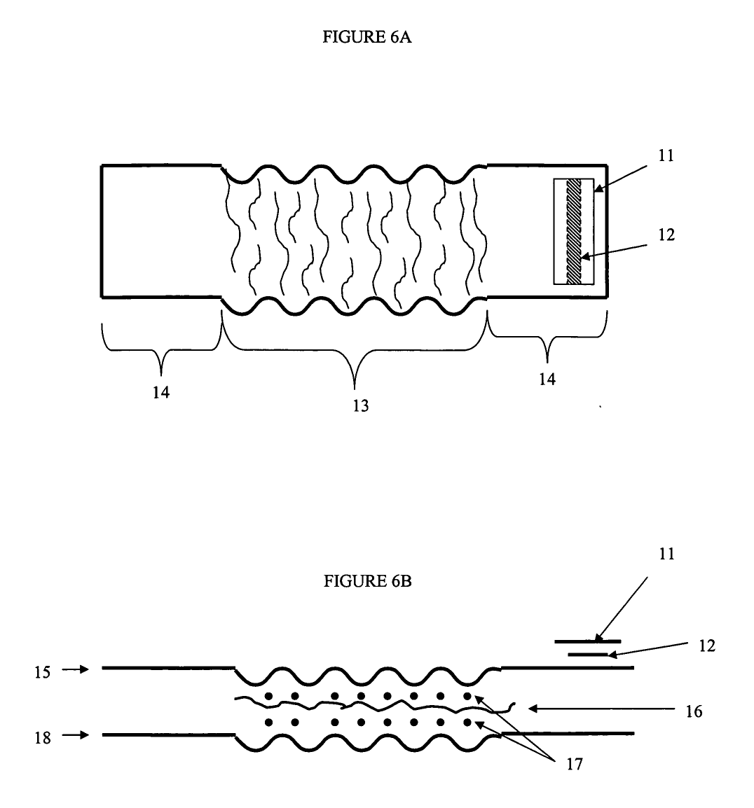

The sheet of the present invention (prior to bonding to the absorbent garment) can also be made of an elastic composite, comprising an elastic material sandwiched in-between two layers of non-woven fabric. The elastic composite can also be bonded to one or more non-stretchable materials in sections (not illustrated). Such elastic composites are commercially available from Clopay Plastic Products of Mason, Ohio. In addition, as shown in FIG. 6A, the sheet can have elastic section 13 and non-elastic section 14. FIGS. 6A and 6B also show a fastener comprising release liner (11) and positioning adhesive (12). FIG. 6B, is a cross section showing how the elastic sheet in FIG. 6A can be constructed with release liner 11, positioning adhesive 12, first sheet of non-woven material 15, one or more elastics 16, adhesive 17, and second sheet 18.

In addition, the sheet of the present invention can also comprise a breathable poly film and non-woven fabric, which can be laminated together. The breathable poly film may comprise a micro-porous film, 2-dimensional apertured film, 3-dimensional apertured film, hydro-entangled film, or any combination thereof. Such 3-dimensional apertured poly films and micro-porous films are commercially available from Tredegar Film Products of Richmond, Va.

An acceptable sheet can also be a combination of layers of the materials listed above. Such combinations can be bonded in various ways, such as by using adhesive, cohesive, ultrasonic bonding, chemical bonding, heat sealing, poly extrusion, etc.

The sheet of the present invention can be various shapes, such as trapezoidal shapes wherein the third edge is wider in length than the fourth edge, or vice versa. In addition, the sheets can be various lengths, but generally a length greater than the width. Furthermore, the width of the sheet should be sufficient, when attached to the absorbent product, to mitigate slippage of the garment causing it to possibly leak. Preferably, the width of the sheet is at least about 4 inches.

The fastener used in the side panel of the present invention can be adapted to affix the side panel with an adhesive such as an adhesive fastening tape. For example, such adhesive fastening tapes are commercially available from 3M Personal Care (3M) of St. Paul, Minn. The adhesive fastening tape may be combined with an adhesive release tape, to create a Y-bond attachment configuration. The adhesive release tape is also commercially available from 3M. In another embodiment, the release liner can be folded over the positioning adhesive.

In addition, the fastener can be adapted to affix the side panel by way of mechanical means. One such mechanical means is a mechanical hook commercially available from 3M. Another commercially available mechanical hook is Velcro®. The use of mechanical hooks requires another material to be attached to, such as a loop (brushed or knit) or some other type of engageable material (ie. as a high loft non-woven). In another embodiment, the adhesive tape and the mechanical attachment system can be combined. Both attachment means can be positioned along side one, another along side two, allowing the user to use either or both.

In a further embodiment, the side panel of the present invention can additionally comprise an adjustable means. The term “adjustable means”, as used herein, is intended to denote a mechanism that allows the side panel's length to be shortened. This can be accomplished by providing a fastener on one side of the panel, then folding the side panel in a Z fold.

In yet a further embodiment, the front portion of the absorbent article can include indicia such as a line or letter (M for medium, L for large, etc.) to indicate where to attach the side panels of the present invention to achieve a particular size circumference.

Without further elaboration the foregoing is intended to be fully illustrative of the present invention so that others may, by applying current or future knowledge, adopt the same for use under various conditions of service.

1. An absorbent article with bonded side panels comprising:
a) an absorbent article having:
i) a front portion having a right edge and left edge;
ii) an absorbent core;
iii) a back portion having a right edge and left edge; and
b) a pair of side panels each comprising:
i) a sheet of skin-friendly material, having:
a first edge and a second edge extending in a lengthwise direction;
a third edge and a fourth edge extending in a widthwise direction; and
ii) a fastener, positioned along the third edge of the side panel, adapted to affix the side panel to the front of the absorbent article,
wherein the fourth edge of each side panel is bonded to the right edge and the left edge of the back portion of the absorbent article, allowing the fastener to be attached to the right edge and the left edge of the front portion of the outside of the absorbent article, securing the absorbent article about the wearer.

2. The bonded side panel of claim 1, wherein the fourth edge is permanently bonded.

3. The bonded side panel of claim 1, wherein all or a portion of the side panel is elastic.

4. The bonded side panel of claim 1, wherein all or a portion of the side panel is a non-woven material.

5. The bonded side panel of claim 1, wherein the third edge has a width greater than the fourth edge or vice versa.

6. The bonded side panel of claim 1, wherein the side panel's length is greater than its width.

7. The bonded side panel of claim 1, wherein the fastener affixes by an adhesive or by a mechanical means, or a combination thereof.

8. The bonded side panel of claim 1, wherein the side panel's width is at least about 4 inches.

9. The bonded side panel of claim 1, wherein the side panel additionally comprises an adjustable means.

10. An absorbent article with bonded side panels comprising:
a) an absorbent article having:
i) a front portion having a right edge and left edge;
ii) an absorbent core;
iii) a back portion having a right edge and left edge; and
b) a pair of side panels each comprising:
iii) a sheet of skin-friendly material, having:
a first edge and a second edge extending in a lengthwise direction;
a third edge and a fourth edge extending in a widthwise direction; and
iv) a fastener, positioned along the third edge of the side panel, adapted to affix the side panel to the front of the absorbent article,
wherein the fourth edge of each side panel is bonded to the right edge and the left edge of the front portion of the absorbent article, allowing the fastener to be attached to the right edge and the left edge of the back portion of the outside of the absorbent article, securing the absorbent article about the wearer.

11. The bonded side panel of claim 10, wherein the fourth edge is permanently bonded.

12. The bonded side panel of claim 10, wherein all or a portion of the side panel is elastic.

13. The bonded side panel of claim 10, wherein all or a portion of the side panel is a non-woven material.

14. The bonded side panel of claim 10, wherein the third edge has a width greater than the fourth edge or vice versa.

15. The bonded side panel of claim 10, wherein the side panel's length is greater than its width.

16. The bonded side panel of claim 10, wherein the fastener affixes by an adhesive or by a mechanical means, or combination thereof.

17. The bonded side panel of claim 10, wherein the side panel's width is at least about 4 inches.

18. The bonded side panel of claim 10, wherein the side panel additionally comprises an adjustable means.

19. An absorbent article with bonded side panels comprising:
c) an absorbent article having:
i) a front portion having a right edge and left edge;
ii) an absorbent core;
iii) a back portion having a right edge and left edge; and
d) a single side panel comprising:
v) a sheet of skin-friendly material, having:
a first edge and a second edge extending in a lengthwise direction;
a third edge and a fourth edge extending in a widthwise direction;
a middle portion positioned between the first, second, third and fourth edges; and
vi) a fastener, positioned along the third edge and middle portion of the side panel, adapted to affix the side panel to the front portion and the back portion of the absorbent article,
wherein the fourth edge of the side panel is bonded to the right edge or the left edge of the back portion of the absorbent article, allowing the fastener to be attached to the front portion and the right or left edge of the back portion securing the absorbent article about the wearer.

20. A method for making an absorbent article with bonded side panels comprising the steps of:
a) preparing a pair of side panels comprising:
i) a sheet of skin-friendly material, having:
a first edge and a second edge extending in a lengthwise direction;
a third edge and a fourth edge extending in a widthwise direction; and
ii) a fastener, positioned along the third edge of the side panel, adapted to affix the side panel to right and left edge of the front or back portion of an absorbent article; and
b) attaching the pair of side panels to a the absorbent article comprising:
i) a front portion having a right edge and left edge;
ii) an absorbent core; and
iii) a back portion having a right edge and left edge,
wherein the fourth edge of the side panels are bonded to the right and left edges of either the front or back portions of the absorbent article in-line during the manufacturing process.
